(19) United States
(12) Patent Application Publication
(10) Pub. No.: US 2003/0053027 A1
(43) Pub. Date: Mar. 20, 2003

(54) Subjective refraction by meridional power matching

(76) Inventor: Edwin J. Sarver, Merritt Island, FL (US)

Correspondence Address: MCHALE & SLAVIN, 4440 PGA BLVD, SUITE 402, PALM BEACH GARDENS, FL, 33410

(21) Appl. No.: 10/124,621

(22) Filed: Apr. 16, 2002

Related U.S. Application Data
(60) Provisional application No. 60/284,435, filed on Apr. 16, 2001.

(51) Int. Cl. A61B 3/10

(52) U.S. Cl. 351/216

(57) ABSTRACT

This invention relates to a low-complexity, low-cost, fast subjective refractor that can measure a patient's required vision correction in terms of sphere, cylinder, and axis using model parameter matching of meridional power values.

### BACKGROUND OF THE INVENTION

1. Field of the Invention

The object of this invention is to realize a low-cost, fast subjective refractor that can measure a patient's required vision correction in terms of sphere, cylinder, and axis using model parameter matching of meridional power values. It is expected that the accuracy and precision of subjective refraction by meridional power matching (SR-MPM) will be similar to subjective refraction using a phoropter or trial lens set. The approach is a technological innovation over traditional methods in that it is low-cost and has low system complexity.

2. Description of the Prior Art

Optometers are optical and mechanical instruments that use subjective, objective, or a combination of subjective and objective techniques used to measure the refractive errors of the eye. A subjective method requires the patient to make some judgment of the quality of the retinal image or focus level. An objective method requires an operator or instrument to examine the light reflected from the retina to make a judgment of the quality of the retinal image or focus level. If the instrument automatically makes an objective judgment, it is referred to as an autorefractor. Bennett [Bennett 89] classifies autorefractors into three main classes depending on the basis of operation. These are analysis of image quality, retinoscopic scanning, and Scheiner disc refraction. Commercial autorefractors representing each of these basic classes are currently available.

The benefits of autorefractors include fast exam times and reasonably reliable refraction for normal patients. Reliability studies of refraction reveal that conventional subjective refraction is reliable to within 0.25 to 0.5 D [Goss 96]. Studies comparing conventional subjective refraction and objective autorefractors indicate that autorefractors are satisfactory for determining a starting point for refraction, but are not satisfactory as a substitute for conventional subjective refraction [Goss 96]. As might be expected, following refractive surgery, autorefractors have difficulty obtaining meaningful results. For example, Salchow, et al, [Salchow 99] report that the objective refraction determined from a commercially available autorefractor delivered erroneous results following refractive surgery, especially after LASIK for hyperopia. Naturally, this limits their use for postoperative care in applications such as enhancement procedures or cataract surgery.

A subjective technique similar in some respects to the SR-MPM approach was employed in the Humphrey Vision Analyzer (HVA) introduced around 1977. The system consisted of a curved mirror, an exotic Alvarez two-element variable-power lens and two sets of cross cylinders. The patient would adjust the spherical correction using the Alvarez lens followed by an adjustment of the cylinder. Iteration of sphere and cylinder adjustment would continue until no more improvement was obtained. Analysis was computed within the instrument based on Humphrey's astigmatic decomposition methods [Bennett 89]. The spherical range of this instrument was 20 D in ⅛-D increments, the cylinder range was 8 D in ⅛-D increments, and the axis was given in 1° increments. Our SR-MPM subjective approach is much simpler than the HVA in terms of operation and optical design. In addition, the SR-MPM units will be much smaller and very low cost.

In another publication, Salmon and Horner [Salmon 96] described a subjective refractor which combined meridional refraction with vernier testing to increase the accuracy of the meridional power measurements and hence the overall accuracy of the system. In their system, a slit target was passed through a split-polarizing filter so that one-half of the slit was linearly polarized in one direction and the other half of the slit was polarized in the orthogonal direction. Another split-polarizing filter, rotated by 90°, was located in front of the eye. The optical effect is to displace the two polarized halves of the slit target if it is out of focus. The power measurements were made in the 180°, 45°, 90° and 135° meridians. The slit target was incorporated within the instrument and the adjustment of the target's axial location by the test subject determined the defocus in a given meridian. Both the target and the polarizers were rotated together for each measurement. As noted by the authors, there was a strong stimulus for proximal accommodation, so cycloplegia was used. To reduce the effect of this problem they suggested a modification to allow fixation of a distant object through a semi-silvered mirror and to provide motorized rather than manual target adjustment. Another major problem was that precise alignment of the subject's eye was critical, and a bite bar was required for stability. In some cases alignment had to be readjusted between meridians. This made the entire procedure tedious and time consuming.

In comparison to the SH system, the SR-MPM approach uses a distant object for distance refraction so that proximal accommodation is not a problem. The precise alignment of the subject's eye and bite bar required by the SH system is not required in our SR-MPM approach since it does not utilize the vernier method. In place of the four meridian positions, multiple power meridians are used for calculations as required. The SR-MPM approach has the ability to use various patterns as targets to compute acuity or contrast sensitivity. This extension is not provided for in the SH system. In a preferred embodiment of the SR-MPM system, the patterns presented to the subject are computer-generated to provide fine control over meridian measurements and pattern resolution. Additionally, by optically moving the test targets to a near-point distance, the SR-MPM can be used for near-point refraction.

### SUMMARY OF THE INVENTION

### BRIEF DESCRIPTION OF THE DRAWINGS

FIG. 1. Basic Block diagram of the SR-MPM. In this figure, we identify the basic elements of the SR-MPM. These are: The stimulus symbol is a pattern viewed by the subject to measure a particular meridional power value. At least three patterns are presented to determine the refraction parameters. The lenses, L1 and L2, relay the view of the symbol and adjust the vergence for the subject. The field lens is an intermediate lens placed slightly off the focal point of L1 (so L2 will not image surface imperfections) to ensure constant magnification of the meridional patterns. The Porro prism pair provides an erect view of the symbol. The aperture stop places the entrance pupil at the first lens so that, in conjunction with the field lens, the system produces constant angular magnification at the exit pupil. The exit pupil is coincident with the entrance pupil of the subject's eye.

FIG. 2. Four bar patterns at orientations of 180, 45, 90, and 135 degrees. Four sample bar patterns are shown for illustrative purposes. In the SR-MPM system, several bar pattern sizes and orientations are used as well as other pattern sizes, orientations, and contrast.

FIG. 3. Simplified optical layout for SR-MPM. In this figure, x1 is the distance from the symbol to lens L1. This distance is nominally 20 feet or 6 meters corresponding to a standard distance for acuity measurement. The derived calculations work for any viewing distance and correspond to the desired correction at that distance. For example, for distance or near correction measurement, the stimulus symbols of the appropriate size are simply placed at the desired distance. The second distance, x′1 is the distance from lens L1 to the aerial image of the symbol. The distance from the aerial image of the symbol to lens L2 is given by the variable value x2. This parameter is adjusted when the subject performs a focusing operation with the SR-MPM unit. The distance from lens L2 to the corneal vertex is given by vd. Note that while this simplified optical layout indicates a simple image rotation, the real implementation employs an image rotation device to erect the image. In addition, a field lens at x1 keeps the magnification constant at the exit pupil.

### DETAILED DESCRIPTION OF THE PREFERRED EMBODIMENTS

This invention relates to a low-cost, fast subjective refractor that can measure a patient's required vision correction in terms of sphere, cylinder, and axis in less than two minutes per eye using model parameter matching of meridional power values. This technique is referred to as subjective refraction by meridional power matching (SR-MPM).

The accuracy and precision of SR-MPM is similar to subjective refraction using a phoropter or trial lens set. The test is quick and simple requiring sequential, one-dimensional searches for best focus on each eye for a small series of test patterns (minimum of 3).

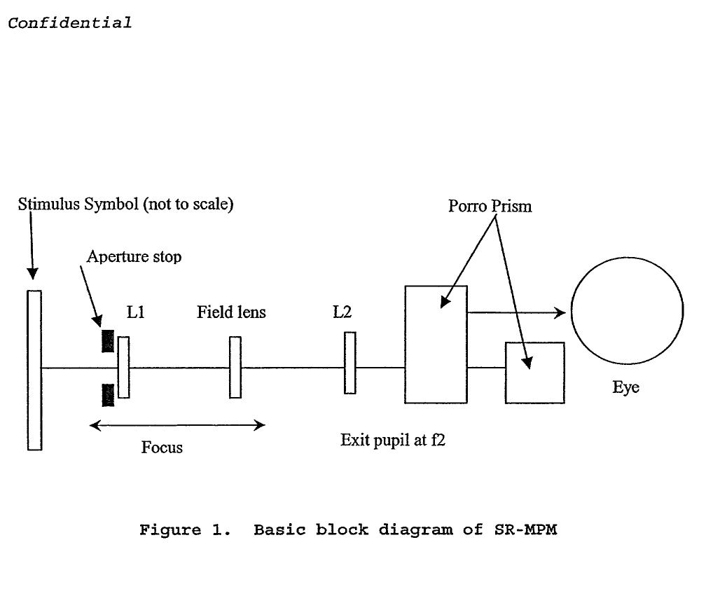

In FIG. 1 the components of the SR-MPM system are identified. The stimulus symbol is a pattern viewed by the subject to measure a particular meridional power value. At least three patterns are presented to determine the refraction parameters. The lenses, L1 and L2, relay the view of the symbol and adjust the vergence for the subject. The field lens is an intermediate lens placed slightly off the focal point of L1 (so L2 will not image surface imperfections) to ensure constant magnification of the meridional patterns. The Porro prism pair provides an erect view of the symbol. The aperture stop places the entrance pupil at the first lens so that, in conjunction with the field lens, the system produces constant angular magnification at the exit pupil.

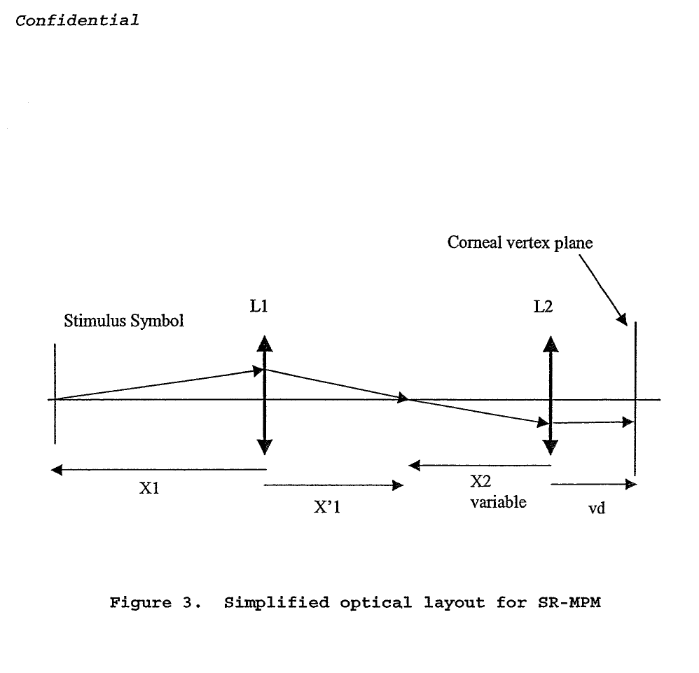

As previously stated, the goal of this simple system is to determine the required “focusing power” (for a discussion about what is and is not in focus see Patterns section below) for the subject to optimally see each of the meridional stimulus patterns. In addition, it is designed to provide constant angular magnification at the exit pupil regardless of defocus. A simplified layout (with prisms and field lens removed) is shown in FIG. 3 below along with the equations for the calculation of refraction. In an alternate embodiment the prisms can be replaced with custom components using first surface mirrors or other image rotation device.

Meridional Stimulus Patterns

To obtain in the meridional power values, computer-generated parallel bar patterns are presented and oriented in the meridian perpendicular to the meridian for which we are obtaining the power measurement [Bennett 78]. For each pattern, the user adjusts the focus arm to best focus for that pattern. It has been noted that refraction data from three or more meridians are sufficient to compute the parameters for spherocylindrical refraction [Salmon 96]. In a preferred embodiment an even number of patterns (at least four) are used, where pattern n is oriented in the n*180/N meridian. In this case, each pattern has a corresponding pattern oriented orthogonal to it. This situation is advantageous because the Euler constant can be used as a quality check on the measured data as explained below. In an alternate embodiment, an odd number of patterns are used or less than three patterns are used as may be allowed for special vision testing requirements. For a given meridian that is not aligned with the principal meridians of a subject's refraction, the patterns will not come to a perfect focus. Thus, in the strictest sense, the traditional definition of power does not exist in an oblique meridian of a spherocylindrical surface. Bennett and Rabbetts [Bennett 76] use the term “notational power” to denote the power in the oblique meridian calculated from the sine square equation.

P_{θ}=S+C sin²(θ−axis) (1)

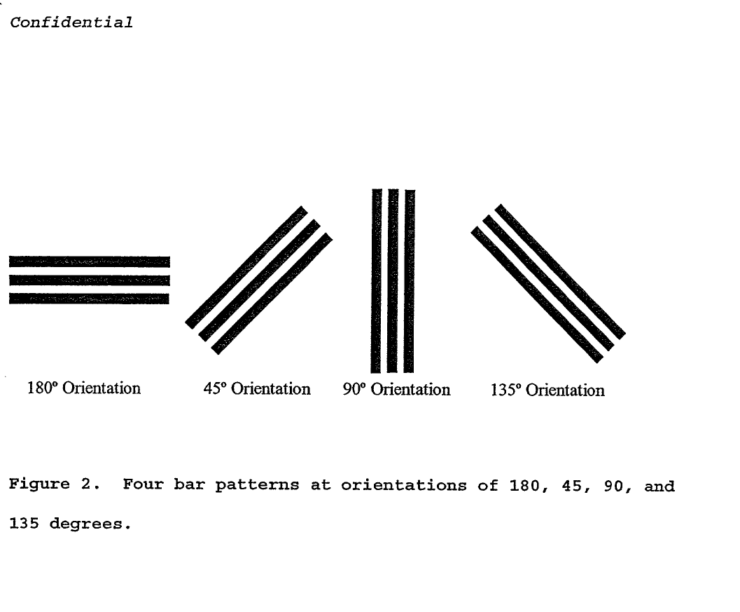

Keating and Carroll [Keating 76] have shown that the sine square law is related to minimizing the blur or maximizing the modulation transfer function for meridional refraction of a line object. This situation has profound implications on the design of the patterns and analysis of the data. The spacing of the bars must be large enough to tolerate the expected defocus in the oblique meridians and prevent spurious resolution during the subject's focusing task. The basic bar patterns for four meridians is illustrated in FIG. 2. Again, the actual meridional power quantities measured by these patterns are in the meridians orthogonal to the pattern orientations. In other embodiments of this invention, other patterns (printed, computer generated, or generated by some other means) with different orientations or contrast are employed.

Calculation of Refraction from Meridional Power Values

The simple optical formulae to perform the desired calculations of focusing power are developed below. The optical diagram for a preferred embodiment of the SR-MPM is given in FIG. 3. In this figure, x1 is the distance from the symbol to lens L1. For distance vision in a preferred embodiment, this distance is typically 20 feet or 6 meters corresponding to a standard distance for acuity measurement. The derived calculations work for any viewing distance and correspond to the desired correction at that distance. For example, for distance or near correction measurement, the stimulus symbols of the appropriate size are simply placed at the desired distance. The second distance, x′1 is the distance from lens L1 to the aerial image of the symbol. The distance from the aerial image of the symbol to lens L2 is given by the variable value x2. This parameter is adjusted when the subject performs a focusing operation with the SR-MPM unit. The distance from lens L2 to the corneal vertex is given by vd. Note that while this simplified optical layout indicates a simple image rotation, the real implementation employs an image rotation device to erect the image. In addition, a field lens at x1 keeps the magnification constant at the exit pupil.

The purpose of the image rotation is to give the user a more comfortable viewing task. For lens L1 we have the relation in equation (2).

$$\frac{1}{{x}^{′} 1} = \frac{1}{x1} + \frac{1}{f1} \qquad ( 2 )$$

The optical power of the image at lens L2 that transmitted to the eye is given in (3).

$$PI = PO + PL \\ PO = \frac{1000}{x2} \\ PL = \frac{1000}{f2} \qquad ( 3 )$$

In equation (3) x2 and the focal length f2 of lens L2 are given in mm. The power at the corneal vertex can now be determined using the effectivity equation in (4).

$${e}_{d , n} ( P ) = \frac{P}{1 - \frac{dP}{n}} \qquad ( 4 )$$

In equation (4), P is the power (in diopters) being translated, n is the index of the medium (times 1000) in which the translation occurs, and d is the distance (in mm) translated. If the translation is to the right (in the direction of light propagation), d is positive. The power at the corneal vertex plane is denoted Pv. Equations (2)-(4) provide the method of computing the meridional power given the system parameters and the position of the linear encoder on the focusing slide. How to compute the refraction components, given a set of meridional power values, is shown next.

Model Fitting

As indicated above, the meridional power for a spherocylindrical refraction is computed using the sine square law, which is given by the following equation (repeated for convenience).

P_{θ}=S+C sin²(θ−axis) (5)

Note that this equation is an approximation since the curvature in a given meridian for a spherocylindrical surface is not constant, but the profile of an ellipse [Keating 88]. The effect of this and other approximations are accounted for in the mapping function described below. In this equation, S denotes the spherical component of refraction in diopters, C is the cylindrical component in diopters, and axis is the orientation meridian of the cylindrical component in degrees. P_{θ}is the meridional power in diopters and θ is the meridian in which the meridional power is given. For example, Pₐₓᵢₛ=S and P_{axis+90°}=S+C.

Now, suppose there are N meridional power measurements, Pₙ, corresponding to a known set of meridians, θₙ. The following system of non-linear equations can be written.

P₀=S+C sin²(θ₀−axis)

P₁=S+C sin²(θ₁−axis)

P_{N-1}=S+C sin²(θ_{N-1}−axis) (6)

There are several methods for solving this system of non-linear equations [Press 92], but in this case the method that appears most robust and still practical is a brute force search over all 180 2×2 linear systems of equations for a given axis value in the range of 1° to 180° in steps of 1°. For a given value of axis, (6) can be written as the following over-determined linear system of equations in (7).

$$[ \begin{matrix}1 & {sin}^{2} ( {θ}_{0} - axis ) \\ 1 & {sin}^{2} ( {θ}_{1} - axis ) \\ ⋮ & ⋮ \\ 1 & {sin}^{2} ( {θ}_{N - 1} - axis )\end{matrix} ] [ \begin{matrix}S \\ C\end{matrix} ] = [ \begin{matrix}{P}_{0}^{′} \\ {P}_{1}^{′} \\ ⋮ \\ {P}_{N - 1}^{′}\end{matrix} ] \qquad ( 7 )$$

The least squares solution to (7) is simply computed using equations (8) (e.g., see [Golub 96]).

$$x = {( {A}^{T} A )}^{- 1} {A}^{T} b \\ [ \begin{matrix}S \\ C\end{matrix} ] = \frac{[ \begin{matrix}c & - b \\ - b & a\end{matrix} ] [ \begin{matrix}e \\ f\end{matrix} ]}{d} \\ Where \\ a = N \\ b = {\sum }_{n = 0}^{N - 1} {sin}^{2} ( {θ}_{n} - axis ) \\ c = {\sum }_{n = 0}^{N - 1} {sin}^{4} ( {θ}_{n} - axis ) \\ d = ac - {b}^{2} \\ e = {\sum }_{n = 0}^{N - 1} {P}_{n} \\ f = {\sum }_{n = 0}^{N - 1} {sin}^{2} ( {θ}_{n} - axis ) {P}_{n} \qquad ( 8 )$$

The error in the system of equations for axis can then be expressed as (9).

$${err}_{axis} = {\sum }_{n = 0}^{N - 1} {( {P}_{n}^{′} - {P}_{n} )}^{2} \qquad ( 9 )$$

The general algorithm for finding the spherocylindrical model parameters for any number N (N>2) of meridional power values can now be stated as follows:

1. Set BestErr to infinity

2. For axis=0 to 179 do the following steps

3. For the current axis value form the system of equations in (7)

4. Solve the equations for S and C using (8)

5. Evaluate the error for this set of S, C, and axis values using (9)

6. If this is the lowest error so far, set BestErr=err, BestS=S, BestC=C, and BestAxis=axis

7. If there are more axis values, continue to step 3, otherwise go to step 8

8. Done, return BestErr, BestS, BestC, and BestAxis

Raw Data Consistency Check

From differential geometry, Euler's law states that for a spherocylindrical surface the sum of any two perpendicular power values is a constant [Keating 88]. This sum is referred to as Euler's constant E and is given by equation (10).

E=[S+C sin²(α)]+[S+C sin²(α+90)] (10)

Since sin²(α+90)=cos²(α),

E=2S+C (11)

The relations given by Euler's constant in (10) and (11) provide the motivation for using orthogonally oriented meridional patterns. For well-behaved refractions, it is expected that the sum of each set of orthogonal meridional pairs is equal. Therefore, these relations can be used to determine the consistency of the measurements by looking at the similarity in the sums of each of the meridional pairs. If the E's for all sets are nearly the same, we can have high confidence in the measurements. If the E's are quite different, we can question the data, and if more than four patterns (two pairs) are used, the probable source of error can be identified. Another cause of unequal E's would be irregular astigmatism, so care must be taken in using and reporting this check. The indicated consistency check is incorporated into the calculations and proves useful in a clinical setting.

Astigmatic Decomposition

In addition to the equations above, the calculations performed by the SR-MPM technique make use of the astigmatic decomposition of a spherocylindrical lens. The astigmatic decomposition transforms the parameters for a spherocylindrical lens into a domain where the components may be linearly combined. Using the formulation in [Bennett 89], the forward astigmatic decomposition is written as indicated in (12).

$$m = sphere + \frac{cyl}{2} \\ {c}_{0} = cyl cos ( axis \times 2 ) \\ {c}_{45} = cyl sin ( axis \times 2 ) \qquad ( 12 )$$

The inverse astigmatic decomposition operation (for plus cylinder notation) is given in (13).

$$cyl = \sqrt{{c}_{0}^{2} + {c}_{45}^{2}} \\ sphere = m - \frac{cyl}{2} \\ axis = {tan}^{- 1} ( \frac{cyl - {c}_{0}}{{c}_{45}} ) \qquad ( 13 )$$

To keep axis between 0 and 180, if the computed value for axis is less than zero, 180 is added to it. The forward and inverse astigmatic decomposition transformations are denoted as in (14) and (15), respectively.

$$\begin{matrix}[ \begin{matrix}m \\ {c}_{0} \\ {c}_{45}\end{matrix} ] = A \{ [ \begin{matrix}sphere \\ cyl \\ axis\end{matrix} ] \} & ( 14 ) \\ [ \begin{matrix}sphere \\ cyl \\ axis\end{matrix} ] = {A}^{- 1} \{ [ \begin{matrix}m \\ {c}_{0} \\ {c}_{45}\end{matrix} ] \} & ( 15 )\end{matrix}$$

Mapping of Measurements to Refractions

Due to the paraxial approximation above and patient psychological factors, the measurements given by the direct application of the equations differ from actual patient refractions. To remedy this situation, a function to map the measurements to reported patient refractions is employed. Let SVM and SVA be two sets of spherocylindrical values for the correction at the corneal vertex plane with the n^{th} values denoted SVM(n) and SVA(n). We want to find a function to estimate SVA(n) given SVM(n). This is indicated in equation (16).

SVA(n)=f(SVM(n)) (16)

Given the measured and actual astigmatic decomposition form, one way to accomplish the mapping indicated in (16) is to use three 3-dimensional P-degree polynomials. That is, use a 3-dimensional P-degree polynomial to estimate the M component of SVA(n), another for the C0 component and a third for the C45 component. This is indicated in equation (17).

$${M}_{R} = {f}_{M} ( \begin{matrix}{M}_{M} \\ {C0}_{M} \\ {C45}_{M}\end{matrix} ) \\ {C0}_{R} = {f}_{C0} ( \begin{matrix}{M}_{M} \\ {C0}_{M} \\ {C45}_{M}\end{matrix} ) \\ {C45}_{R} = {f}_{C45} ( \begin{matrix}{M}_{M} \\ {C0}_{M} \\ {C45}_{M}\end{matrix} ) \qquad ( 17 )$$

Each of the mapping functions f_{M}, f_{C0}, f_{C45} in (17) is a 3-dimensional P-degree polynomial.

N-Dimensional, M-Degree Polynomials

The mapping functions employed are polynomials. Polynomials can be of varying degree and dimension. The form of the N-dimensional, M-degree polynomial is given by (18).

$$\begin{matrix}f ( {x}_{0} , {x}_{1} , … , {x}_{N - 1} ) = {\sum }_{{i}_{N - 1} = 0}^{M} … {\sum }_{{i}_{1} = 0}^{{i}_{2}} {\sum }_{{i}_{0} = 0}^{{i}_{1}} {c}_{{i}_{0} , {i}_{1} , … {i}_{N - 1}} {x}_{0}^{{i}_{0}} {x}_{1}^{{i}_{1} - {i}_{0}} … \\ {x}_{N - 1}^{{i}_{N - 1} - {i}_{N - 2}}\end{matrix} \qquad ( 18 )$$

where

| Parameter | Description |
|---|---|
| N | dimension of polynomial |
| M | order of polynomial |
| cᵢ_{₀}_{,i}_{₁}_{,...i}_{_{N-1}} | coefficients for polynomial terms |

Three-Dimensional, Low-Order Polynomial Examples

Three-Dimensional, Order-one Polynomial (N=3, M=1)

$$\begin{matrix}f ( {x}_{0} , {x}_{1} , {x}_{2} ) = {\sum }_{{i}_{2} = 0}^{1} {\sum }_{{i}_{1} = 0}^{{i}_{2}} {\sum }_{{i}_{0} = 0}^{{i}_{1}} {c}_{{i}_{0} , {i}_{1}} {x}_{0}^{{i}_{0}} {x}_{1}^{{i}_{1} - {i}_{0}} {x}_{2}^{{i}_{2} - {i}_{1}} \\ = {c}_{0 , 0 , 0} + {c}_{0 , 0 , 1} {x}_{2} + {c}_{0 , 1 , 1} {x}_{1} + {c}_{1 , 1 , 1} {x}_{0}\end{matrix}$$

Three-Dimensional, Order-two Polynomial (N=3, M=2)

$$\begin{matrix}f ( {x}_{0} , {x}_{1} , {x}_{2} ) = {\sum }_{{i}_{2} = 0}^{2} {\sum }_{{i}_{1} = 0}^{{i}_{2}} {\sum }_{{i}_{0} = 0}^{{i}_{1}} {c}_{{i}_{0} , {i}_{1}} {x}_{0}^{{i}_{0}} {x}_{1}^{{i}_{1} - {i}_{0}} {x}_{2}^{{i}_{2} - {i}_{1}} \\ = {c}_{0 , 0 , 0} + {c}_{0 , 0 , 1} {x}_{2} + {c}_{0 , 1 , 1} {x}_{1} + {c}_{1 , 1 , 1} {x}_{0} + {c}_{0 , 0 , 2} {x}_{2}^{2} + \\ {c}_{0 , 1 , 2} {x}_{1} {x}_{2} + {c}_{1 , 1 , 2 ,} {x}_{0} {x}_{2} + {c}_{0 , 2 , 2} {x}_{1}^{2} + {c}_{1 , 2 , 2} {x}_{0} {x}_{1} + {c}_{2 , 2 , 2} {x}_{0}^{2}\end{matrix}$$

Fitting to N-Dimensional Data

To fit the polynomial to N-dimensional data, the standard square system

A^{T}Ax=A^{T}b

is formed, where the dimensions of A is K×N, x is N×1 and b is K×1 and K is the number of constraints on the system. Each row of A contains the products of the variable powers corresponding to the coefficients. Each row of b contains the desired value of the polynomial at the given location. The corresponding rows of A and b can be multiplied by a weight to yield a weighted least squares fit.

Building A^{T}A And A^{T}B

Rather than directly building the A matrix and b vector and then computing A^{T}A and A^{T}b, they can be built incrementally using outer products. A weight may be added to the constraint just as in the normal case of building A and b. The resulting system of equations can be solved using a number of algorithms. Two particularly good algorithms for this case are the singular value decomposition (SVD) [Golub 96] or the Cholesky algorithm [Press 92].

Use of Focus Position Signal

During the subject's task of focusing on a given pattern, the linear encoder detects the location of the focus mechanism so that the software can sample the subject's current focusing power as a function of time. Analysis of this discrete sequence can be used to further improve the accuracy of the meridional power measurements. Analysis of this discrete sequence can also be used to estimate the confidence of in the meridional power measurement.

Other Embodiments

In addition to the embodiments above, the SR-MPM could be configured as a hand-held instrument or supported by a simple support or table. In addition to the patterns being generated on printed matter such as a poster or generated on a computer screen, the patterns could also be generated by projection, reflection, or any other such means. In addition to the patterns being external to the SR-MPM unit at any distance, the patterns could be incorporated within the SR-MPM unit or some patterns could be generated outside the SR-MPM and some within the SR-MPM unit. In addition to the simple optical layout, more complex lenses (for example to decrease optical aberrations) could be employed to measure the meridional power. The SR-MPM unit could be controlled directly by a host computer such as a PC, or could be controlled by an embedded processor or a combination of the two. The SR-MPM unit could be connected directly to a printer or display device or network or could be interfaced to a host computer such as a PC to print, display, and store exams. A printer and display device could also be made part of the SR-MPM unit. A mechanical only version of the SR-MPM could be used in very harsh environments. In such a case a table or set of tables or a curve or set of curves could be used to convert measured meridional powers indicated by the slide position into the desired sphere, cylinder, and axis values. In addition, it is possible to compute higher order aberrations above sphere, cylinder, and axis value using proper surface fitting techniques. These higher order aberrations could be represented as Zernike polynomial coefficients. The SR-MPM could be supplied with power from an external source or battery operated.

Literature Cited

[Bennett 76] Bennett A G, Rabbetts P B, “Refraction in Oblique Meridians of the Astigmatic Eye,” Brit. J. Physiol. Opt. 1976;32;59-77.

[Bennett 78] Bennett A G, “Methods of automated objective refraction,” Ophthalmic Optician 1978;8(1):8-13,19.

[Bennett 89] Bennett A G, Rabbetts, R B, Clinical Visual Optics, Second Edition, Butterworth-Heinemann Ltd, 1989.

[Elmstrom 77] Elstrom G, “Management of considerations of the Humphrey Vision Analyzer,” J Am Optom Assoc October 1977; 48(10);1293-1299.

[Fannin 87] Fannin T E, Grosvenor T P, Clinical Optics, Butterworth-Heinemann, 1987.

[Golub 96] Golub G H, Van Loan C F, Matrix Computations, Third Edition, The John Hopkins University Press, 1996.

[Goss 96] Goss D A, Grosvenor T, “Reliability of refraction—a literature review,” J Am Optom Assoc October 1996;67(10)619-630.

[Keating 76] Keating M P and Carroll, Blurred Imagery and the Sine Squared Law, Amer. J. Optom. Physiol. Opt. 1976;53; 66-69.

[Keating 88] Keating M P, Geometric, Physical, and Visual Optics, Butterworth-Heinemann, 1988.

[Liang 94] Liang J, Grimm B, Goelz S, Bille J F, “Objective measurement of wave aberrations of the human eye with the use of a Hartmann-Schack wave-front sensor,” J Opt Soc Am A 1994;11(7);1949-1957.

[Press 92] Press W H, Teukolsky S A, Vetterling W T, Flannery B P, Numerical Recipes in C: The Art of Scientific Computing, Second Edition, Cambridge University Press, 1992.

[Salchow 99] Salchow D J, Zirm M E, Stieldorf C, Parisi A, “Comparison of objective and subjective refraction before and after laser in situ keratomileusis,” J Cataract Refract Surg June 1999;25(6);827-835.

[Salmon 96] Salmon TO, Horner DG, “A new subjective refraction method—the meridional polarized vernier optometer,” J Am Optom Assoc October 1996;67(10);599-605.

[Smith 97] Smith G, Atchison D A, The Eye and Visual Optical Instruments, Cambridge University Press, 1997.

[Thibos 97] Thibos L N, Wheeler W, Horner D G “Power vectors: an application of Fourier analysis to the description and statistical analysis of refractive error,” Optom Vis Sci 1997;74;367-375.

[Thibos 01] Thibos L N, Horner D G, “Power vector analysis of the optical outcome of refractive surgery,” J Cataract Refract Surg 2001;27;80-85.

I claim:

1. The method of diagnosing a patient's required vision correction in sphere, cylinder and axis using subjective refraction by meridional power matching comprising the steps of:
a) providing an optical instrument having a viewfinder, a first lens, a second lens, a field lens, an aperture stop, a stimulus symbol means having a plurality of patterns for viewing in different meridians, and a focusing means for manipulating the view of said stimulus symbol means;
b) placing said stimulus symbol means at a given distance from said first lens;
c) the patient sequentially viewing each of said plurality of patterns;
d) the patient adjusting said focusing means to focus each of said plurality of patterns;
e) determining the optical distance between said second lens and each of said plurality of patterns at focus c); and
f) calculating the meridional power values.

2. The method of claim 1 further measuring distant refraction by placing said stimulus symbol means at a first commensurate distance from said first lens.

3. The method of claim 2 further measuring near point refraction by optically moving said stimulus symbol means from said first commensurate distance to a commensurate near point distance from said first lens.

4. The method of claim 1 further orienting one of each of said plurality of patterns in one of at least three meridians.

5. The method of claim 1 further providing an image of said stimulus symbol and rotating said image optically.

6. The method of claim 5 further providing means to erect said image optically.

7. The method of claim 1 further timing said adjusting of said focusing means and analyzing the discrete sequence to improve accuracy of the meridional power measurements.

8. The method of claim 1 further providing said optical instrument with computer control, electronically generating said stimulus symbol means and calculating said meridional power measurements by algorithms.

9. A subjective refractor for diagnosing vision correction in sphere, cylinder and axis comprising an optical instrument having a viewfinder, a first lens, a second lens, an aperture stop, a stimulus symbol means having a plurality of patterns for viewing in different meridians, and a focusing means for manipulating the focus of said stimulus symbol means operatively connected together, wherein said first lens and said second lens relay the image of said stimulus symbol means to the eye, said plurality of patterns placed at a particular distance from said first lens, said second lens movable in relation to said first lens and said plurality of patterns, said focusing means including a focus arm means for moving said second lens and focusing said plurality of patterns, said location of said focus arm denoting the distance between said second lens and said plurality of patterns whereby said focus arm gives a distance measurement when said plurality of patterns are focused and said distance measurement results in a particular meridional power value.

10. A subjective refractor of claim 9 further comprising a field lens intermediate said first and said second lens for providing constant magnification of said plurality of patterns.

11. A subjective refractor of claim 10 further comprising said plurality of patterns each being oriented in a different meridian.

12. A subjective refractor of claim 11 further comprising a device for optically erecting said image in said instrument.

13. A subjective refractor of claim 12 further comprising a prism.

14. A subjective refractor of claim 9 further comprising a computer and said distance measurement is input into software which solves for meridional power value.
